(19) United States
(12) Patent Application Publication
(10) Pub. No.: US 2005/0096722 A1
(43) Pub. Date: May 5, 2005

(54) Stent implantation system and vessel support

(75) Inventors: Daniel Lootz, Warnemeude (DE); Stephane Delaloye, Buelach (CH); Curt Kranz, Berlin (DE); Goran Lukic, Bulach (CH)

Correspondence Address: HAHN LOESER & PARKS, LLP, One GOJO Plaza, Suite 300, AKRON, OH, 44311-1076, US

(73) Assignee: BIOTRONIK GmbH & Co. KG

(21) Appl. No.: 10/892,528

(22) Filed: Jul. 14, 2004

(30) Foreign Application Priority Data
Jul. 17, 2003 (DE) 103 33 511.0

(51) Int. Cl. A61F 2/06

(52) U.S. Cl. 623/1.11

(57) ABSTRACT

A stent implantation system comprising a catheter and an expandable vessel support which is releasable fixed in the region of the distal end thereof and which has a carrier structure which encloses a cavity open at its distal ends characterized in that the carrier structure of the vessel supports formed by at least two carrier rings which in their non-expanded condition are jointly fixed to the catheter and are not connected together otherwise.

### BACKGROUND OF THE INVENTION

The invention concerns a stent implantation system and an intraluminal vessel support, in particular in the form of a stent.

Stent implantation systems of that kind (referred to as a stent delivery system, for brevity SDS) usually have a catheter, in particular a balloon catheter, on which a vessel support, in particular a stent, is fixed, usually in the region of the distal end of the catheter, as long as the vessel support or stent is not yet implanted and expanded.

For treating, in particular, vessel constrictions in human blood vessels, referred to as stenoses, a stent or a vessel support is usually guided intraluminally to the location of a vessel constriction by means of a balloon catheter. At that location, the stent or vessel support is expanded or will cause itself to expand. After the vessel support or stent has been positioned and expanded, the catheter serving to introduce the stent and the stent itself are separated from each other and the catheter can be removed.

The vessel support or the stent usually have a carrier structure which is mostly of a lattice-like configuration and they generally have a plurality of interconnected metal bars as carrying components. The carrier structure usually encloses a generally cylindrical cavity which is open at its two ends. By virtue of folding of the bars forming the carrier structure, the carrier structure can usually be expanded in the radial direction and can assume a non-expanded compressed condition and from that condition can be converted into an expanded condition of larger diameter.

In that respect, a distinction is usually drawn between balloon-expandable stents and self-expanding stents. Balloon-expandable stents must be expanded, for example, by means of an expandable balloon, into which a fluid is pumped by way of a lumen of the catheter, while self-expanding stents, by virtue of spring forces inherent in the stent, already have a tendency to assume an expanded condition without being assisted by a balloon in that respect. Self-expanding stents frequently have for that purpose, a carrier structure which is formed substantially by a memory metal such as, for example, Nitinol. Such a memory metal has the property of retaining a first shape below a given temperature, referred to as the transformation temperature, and assuming a second shape when the transformation temperature is exceeded. In relation to stents, the first shape corresponds to the compressed condition of the stent and the second shape corresponds to the expanded condition of the stent.

In the case of stent implantation systems, in particular for balloon-expandable stents, but also for self-expanding stents, a balloon is usually arranged in the region of the distal end of the respective catheter, the balloon being in fluid communication by way of a lumen with the proximal end of the catheter so that the balloon can be expanded by introducing a fluid from the proximal end of the catheter.

The vessel support or the stent is usually crimped onto the compressed balloon in such a way that the compressed stent is of the smallest possible outside diameter and is sufficiently firmly secured to the catheter. That permits the stent to be introduced into a blood vessel in a manner which is as simple and secure and reliable as possible, as far as the respective implantation location, for example a coronary blood vessel. At the implantation location the stent—depending on whether it is self-expanding or not—is expanded with or without the aid of the balloon to such an extent that the blood vessel is of a sufficient inside diameter by means of the stent at the location to be treated. The catheter with the balloon can then be removed while the stent remains in the vessel to be treated.

In this connection, some properties of the stent are to be noted. Firstly, the stent must be designed in such a way that it applies to the blood vessel a radial force which is sufficient to hold the blood vessel open. In addition, particularly in connection with balloon-expandable stents, it is to be noted that, in particular when they comprise metal, they involve both a plastic and also an elastic deformation component, with the consequence that, after expansion of the stent, the stent experiences a spring-back effect, referred to as the recoil. This means that the stent has to be enlarged beyond its definitive diameter, in a manner which is basically undesirable. Furthermore, although the stent is intended to support the respective blood vessel in a radial direction, it is however to be as flexible as possible in the longitudinal direction so that the supported blood vessel remains as flexurally soft as possible, in relation to its longitudinal direction.

The various desirable properties of stents usually necessitate compromises in the design of the stent and the stent implantation system.

### SUMMARY OF THE INVENTION

The object of the invention is to provide a stent implantation system and a vessel support which, as far as possible, avoid the disadvantages of the state of the art.

In accordance with the invention, that object is attained by a stent implantation system comprising a catheter and an expandable vessel support which is releasably fixed in the region of the end thereof and which has a carrier structure which encloses a cavity open at its ends and is formed by at least two carrier rings which in, their non-expanded (compressed) condition, are jointly fixed to the catheter and are not connected together otherwise so that after release from the catheter, for example, after an implantation procedure, the carrier rings are independent of each other and are not joined together.

After implantation, the relative position of the mutually independent carrier rings is thus no longer afforded by connecting members between the individual carrier rings but for example solely by a wall of a blood vessel, against which the carrier rings which are expanded after implantation jointly bear.

In contrast to known stents, the individual carrier rings of the stent according to the invention are not connected together by bars, wires or threads. The carrier rings are also not connected by textile fabric or by films. Such films or textile fabric are for example occasionally provided in so-called grafts which however, in contrast to stents, do not serve to remove a vessel constriction but to remove vessel lesions, that is to say in a way to repair leaky blood vessels.

The object of the invention is further attained by a carrier ring for such a stent implantation system or such a vessel support, which is formed by at least one bar extending in the peripheral direction and folded at least in the non-expanded condition with respect to the peripheral direction, wherein the folding allows expansion in the radial direction and in the peripheral direction of the carrier ring.

In a preferred variant, the catheter of the stent implantation system has positioning elements which are arranged between the carrier rings and are adapted to establish the relative position of the carrier rings in the non-expanded condition thereof. The positioning elements, therefore, prevent in particular, slipping of the carrier rings with respect to the longitudinal direction of the catheter.

In addition, the catheter preferably has an expandable balloon which is of such an arrangement and configuration that at least the carrier ring is expandable with the balloon. In a variant the catheter has precisely one balloon, on the outside of which the carrier rings are arranged in the non-expanded condition. In this embodiment, the positioning elements can be positioning rings which are arranged either within the balloon or on the outside of the balloon between the carrier rings. The diameter of the positioning rings preferably approximately corresponds to the diameter of the carrier rings in their compressed (non-expanded) condition.

In an alternative, also advantageous embodiment, the catheter has a plurality of balloons which are of such a configuration and arrangement that individual carrier rings are expandable independently of each other by means of a respective balloon. In that arrangement, the balloons can each be connected by way of their own lumen to a proximal end of the catheter so that each balloon is to be expanded by the introduction of a fluid into the corresponding lumen, independently of the other balloons. It is however, also possible to provide a common lumen for a plurality of balloons, in which case a valve which is to be selectively closed and opened is provided between the lumen and the respective balloon so that an individual balloon can be specifically expanded by closing or opening the respective valve.

A stent implantation system is also preferred in which a carrier ring is formed by at least one bar extending in the peripheral direction and folded at least in the non-expanded condition with respect to the peripheral direction, wherein the folding of the bar allows expansion in the radial direction and in the peripheral direction of the carrier ring.

In a particularly simple alternative configuration, each carrier ring of the stent implantation system is formed by precisely one bar extending in the peripheral direction.

As an alternative thereto, the carrier ring may also have a plurality of expansion cells which are arranged in succession in the peripheral direction and which are each formed by bars enclosing a free space and are connected together to form a closed carrier ring in the peripheral direction by at least one respective connecting bar. The last-mentioned variant of a carrier ring with expansion cells affords an increased support force on the part of the carrier ring in the expanded condition, in comparison with the above-indicated simple variant of the invention.

In that respect, the expansion cells are preferably shaped in such a way that, starting from the non-expanded condition of the carrier ring, they allow radial expansion of the carrier ring. Preferably, the expansion cells are connected together in the peripheral direction of the carrier ring by precisely one respective connecting bar.

In alternative variants of the expansion cells, they are for example, of a symmetrical or asymmetrical configuration with respect to the peripheral direction of the carrier ring. In a particularly preferred, asymmetrical variant, the expansion cells are of a keyhole-like configuration. That preferred variant will be discussed in greater detail with reference to the embodiment described by way of example hereinafter.

Suitable and respectively preferred materials for the carrier rings are metals such as in particular stainless steel (L 360) or also magnesium for balloon-expandable carrier rings. A suitable material for self-expanding carrier rings is Nitinol. Preferably, the bars of a respective carrier ring have one of the above-mentioned metals and are coated on their outside, for example, with silicon carbide or with a polymer or with a drug or with a combination of those materials.

In addition, in a preferred stent implantation system, at least one of the carrier rings has at least one marker element of X-ray-opaque material. X-ray-opaque materials are, for example, metals with a high linear fluctuation coefficient such as gold, tantalum or platinum. The marker element can be, for example, in the form of a complete or partial coating on a carrier ring with an X-ray-opaque material such as gold, tantalum or platinum.

A preferred variant of the invention will now be described in greater detail by means of the embodiment by way of example thereof illustrated in the Figures in which:

### BRIEF DESCRIPTION OF THE DRAWINGS

FIG. 1a shows a side view of a portion of a stent implantation system,

FIG. 1b shows a view in section of the portion shown in FIG. 1a,

FIG. 2 shows a view in section of a portion of an alternative embodiment of a stent implantation system,

FIG. 3 shows a particularly simple variant of a support ring,

FIG. 4a shows a side view of a portion of an alternative stent implantation system, and

FIG. 4b shows a view on an enlarged scale of a portion of the stent implantation system from FIG. 4a.

### DETAILED DESCRIPTION OF THE INVENTION

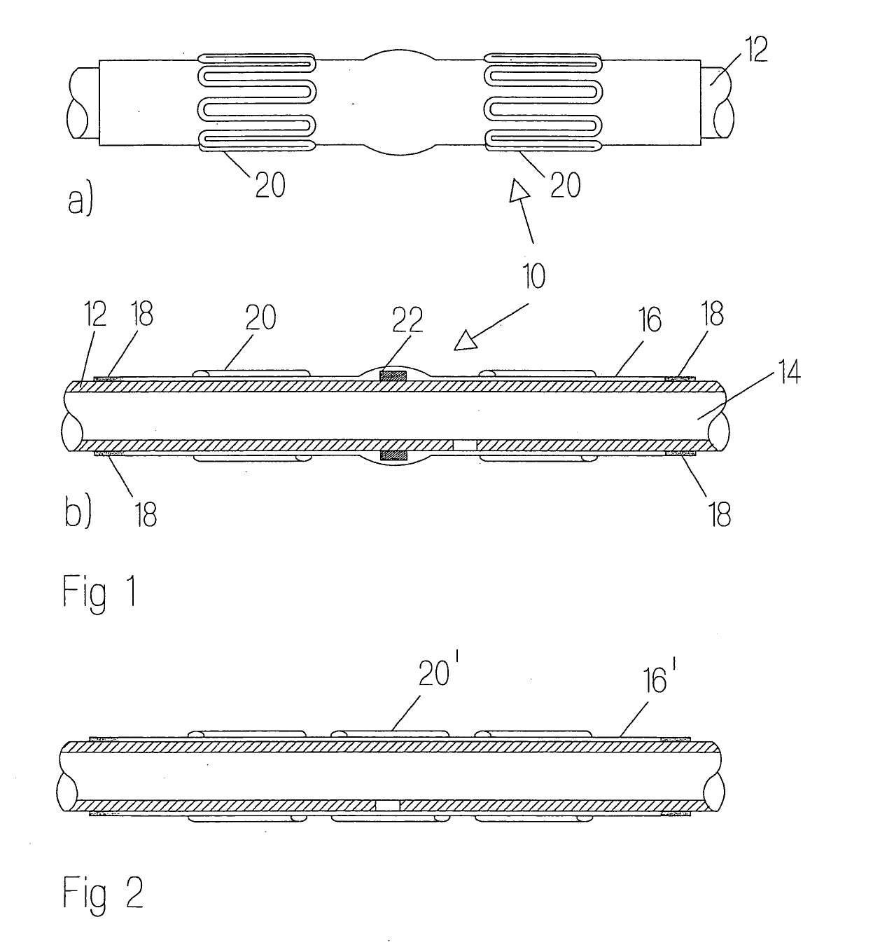

The portion of a stent implantation system which is shown in FIGS. 1a and 1b comprises a catheter shaft 12 having at least one lumen 14 for introducing fluid into a balloon 16. The balloon 16 is connected to the catheter shaft 12 in sealed relationship at weld locations 18. On its outside, the balloon 16 carries carrier rings 20 which together form a vessel support. The carrier rings 20 are crimped onto the balloon 16 which is shown in FIGS. 1a and 1b in the compressed, that is to say, non-expanded condition.

As can be clearly seen, the carrier rings 20 are connected together exclusively by the balloon 16 onto which the carrier rings 20 are crimped. The consequence of this is that, after expansion and subsequent deflation of the balloon, the carrier rings 20 are released therefrom and thereafter are completely independent of each other and are not joined together.

Arranged between the carrier rings 20 in the axial direction of the shaft 12 within the balloon 16 is a positioning ring 22 which ensures that the carrier rings 20 crimped on the balloon 16 cannot slip towards each other to such an extent that for example they hook onto each other. Instead of a single positioning ring 22 it is also possible to provide a plurality thereof, for example in proximal and distal relationship with the carrier rings 20 so that the carrier rings 20 are prevented from slipping off the balloon 16.

After the stent implantation system, together with the carrier rings 20, has been moved to a desired position within a blood vessel to be treated, the balloon 16 can be expanded by introducing fluid into the lumen 14. As a result, the carrier rings 20 are also radially enlarged, with plastic deformation thereof, so that they then retain their larger diameter and periphery. After expansion, the balloon 16 can be deflated again so that the carrier rings 20 come lose from the balloon 16 and the catheter shaft 12 together with the balloon 16 can be removed from the blood vessel. The expansion effect provides that in the implantation procedure, the carrier rings 20 are pressed against the wall of a blood vessel to be treated and are held at the implantation location by the vessel wall when finally the catheter shaft 12 together with balloon 16 are removed. There is then no longer any technical connection in another fashion between the individual carrier rings 20.

FIG. 2 shows an alternative embodiment of a portion of a stent implantation system in which there are no positioning rings provided, for fixing the carrier rings 20′ on the balloon 16′. Instead, the longitudinal ends of the carrier rings 20′ are crimped onto the balloon 16′ more strongly than interposed bar portions of the carrier rings 20′.

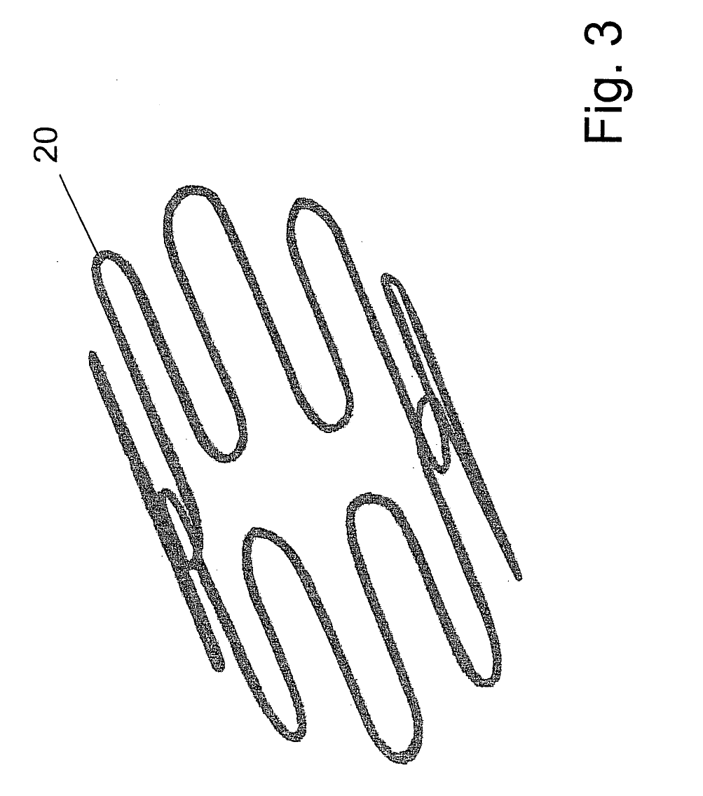

FIG. 3 shows a perspective view on an enlarged scale of a particularly simple carrier ring corresponding to the carrier rings 20 shown in FIG. 1.

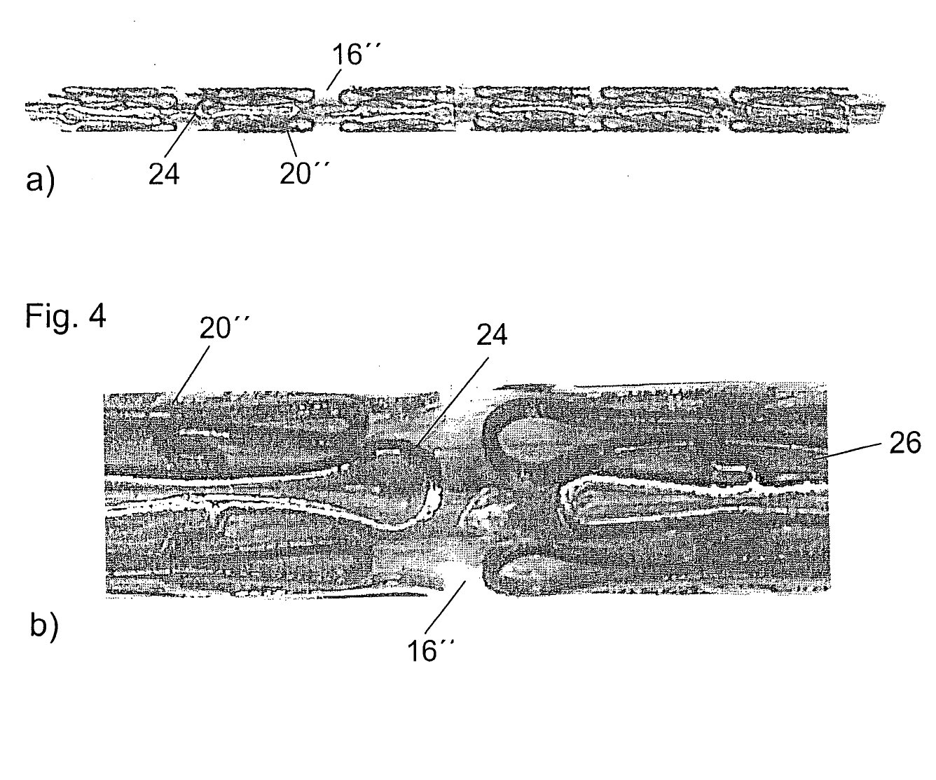

FIG. 4 shows portions of an alternative variant. In this case the catheter with the balloon 16″ corresponds to the structure shown in FIG. 1. In contrast, the carrier rings 20″ are of a different configuration from FIGS. 1 through 3. The carrier rings embrace expansion cells 24 formed by a bar enclosing a free space. Those expansion cells 24 are each connected together by precisely one respective connecting bar 26 in the peripheral direction of a carrier ring 20″ to form a closed carrier ring.

It applies to all illustrated variants that the carrier rings 20, 20′ and 20″ are crimped onto the respective balloon 16 or 16′ in the non-expanded, compressed condition, and are only connected together by the balloon catheter. After expansion, the carrier rings can come lose from the balloon catheter and are independent of each other. The result of this is that flexural mobility of the treated blood vessels is only minimally restricted.

In addition, it is basically to be noted that variants similar to that shown in FIG. 4 are preferred, in which the individual carrier rings on the respective balloon are only at a small axial spacing from each other, unlike the situation shown for example in FIGS. 1 and 2.

Finally, the carrier rings according to the invention differ from conventional known stents in that they are formed at least over parts of their periphery only by a single bar. That is apparent in regard to the carrier rings 20 and 20′. In the case of the carrier rings 20″ in FIG. 4, the sole bar extending in the peripheral direction is the respective connecting bar 26 between the expansion cells 24.

Structural variants in which, instead of a balloon, there a plurality of balloons which, depending on the respective wishes involved, can be inflated and thus expanded independently of each other, are not illustrated in the Figures but can be easily derived from the Figures. For that purpose, a plurality of separate lumens can be provided in the catheter shaft. Alternatively it is also possible to provide a common inflation lumen which, in the region of the distal end, has a plurality of openings which are to be closed or opened in the manner of a valve and which are arranged between the inflation lumen and the respective balloon.

1. A stent implantation system comprising a catheter and an expandable vessel support which is releasably fixed in the region of the distal end thereof and which has a carrier structure which encloses a cavity open at its ends characterized in that the carrier structure of the vessel support is formed by at least two carrier rings, which in their non-expanded condition, are jointly fixed to the catheter and are not connected together otherwise.

2. A stent implantation system as set forth in claim 1 wherein the catheter has positioning elements which are arranged between the carrier rings and are adapted to establish the relative position of the carrier rings in the non-expanded condition thereof.

3. A stent implantation system as set forth in claim 1 wherein the catheter has an expandable balloon which is of such an arrangement and configuration that at least one of the carrier rings is expandable with the balloon.

4. A stent implantation system as set forth in claim 3 wherein the catheter has one balloon, on the outside of which the carrier rings are arranged in the non-expanded condition.

5. A stent implantation system as set forth in claim 3 wherein the catheter has a plurality of balloons which are of such a configuration and arrangement that individual carrier rings are expandable independently of each other by means of a respective balloon.

6. A stent implantation system as set forth in claim 1 wherein a carrier ring is formed by at least one bar extending in the peripheral direction and folded at least in the non-expanded condition with respect to the peripheral direction, wherein the folding of the bar allows expansion in the radial direction and in the peripheral direction of the carrier ring.

7. A stent implantation system as set forth in claim 6 wherein the carrier ring is formed by precisely one bar extending in the peripheral direction.

8. A stent implantation system as set forth in claim 6 wherein the carrier ring has expansion cells which are arranged in succession in the peripheral direction and which are each formed by bars enclosing a free space and are connected together to form a closed carrier ring in the peripheral direction by at least one respective connecting bar.

9. A stent implantation system as set forth in claim 8 wherein the expansion cells are shaped in such a way that, starting from the non-expanded condition of the carrier ring, they allow radial expansion of the carrier ring.

10. A stent implantation system as set forth in claim 8 wherein the expansion cells are connected together in the peripheral direction of the carrier ring by precisely one respective connecting bar.

11. A carrier ring for a stent implantation system comprising:
at least one bar extending in the peripheral direction and folded at least in a non-expanded condition with respect to a peripheral direction, wherein the folding allows expansion in a radial direction and in the peripheral direction of the carrier ring.

12. A carrier ring as set forth in claim 11 wherein the carrier ring is formed by precisely one bar extending in the peripheral direction.

13. A carrier ring as set forth in claim 11 wherein the carrier ring has expansion cells which are arranged in succession in the peripheral direction and which are each formed by bars enclosing a free space and are connected together to form a closed carrier ring in the peripheral direction by at least one respective connecting bar.

14. A carrier ring as set forth in claim 13 wherein the expansion cells are shaped in such a way that, starting from the non-expanded condition of the carrier ring, they allow radial expansion of the carrier ring.

15. A carrier ring as set forth in claim 13 wherein the expansion cells are connected together in the peripheral direction of the carrier ring by precisely one respective connecting bar.

16. A carrier ring as set forth in claim 14, wherein the expansion cells are connected together in the peripheral direction of the carrier ring by precisely one respective connecting bar.

17. A stent implantation system as set forth in claim 9, wherein the expansion cells are connected together in the peripheral direction of the carrier ring by precisely one respective connecting bar.

18. A stent implantation system as set forth in claim 2, wherein a carrier ring is formed by at least one bar extending in the peripheral direction and folded at least in the non-expanded condition with respect to the peripheral direction, wherein the folding of the bar allows expansion in the radial direction and in the peripheral direction of the carrier ring.

19. A stent implantation system as set forth in claim 3, wherein a carrier ring is formed by at least one bar extending in the peripheral direction and folded at least in the non-expanded condition with respect to the peripheral direction, wherein the folding of the bar allows expansion in the radial direction and in the peripheral direction of the carrier ring

20. A stent implantation system as set forth in claim 4, wherein a carrier ring is formed by at least one bar extending in the peripheral direction and folded at least in the non-expanded condition with respect to the peripheral direction, wherein the folding of the bar allows expansion in the radial direction and in the peripheral direction of the carrier ring.

21. A stent implantation system as set forth in claim 5, wherein a carrier ring is formed by at least one bar extending in the peripheral direction and folded at least in the non-expanded condition with respect to the peripheral direction, wherein the folding of the bar allows expansion in the radial direction and in the peripheral direction of the carrier ring.
